(19) United States
(12) Patent Application Publication
(10) Pub. No.: US 2005/0238179 A1
(43) Pub. Date: Oct. 27, 2005

(54) Active noise reduction in the proximity of a passenger seat

(76) Inventor: Wolfgang Erdmann, Buxtehude (DE)

Correspondence Address: OSTROLENK FABER GERB & SOFFEN, 1180 AVENUE OF THE AMERICAS, NEW YORK, NY, 100368403, US

(21) Appl. No.: 11/112,791

(22) Filed: Apr. 21, 2005

Related U.S. Application Data
(60) Provisional application No. 60/600,062, filed on Aug. 9, 2004.

(30) Foreign Application Priority Data
Apr. 23, 2004 (DE) 10 2004 019 788.1

(51) Int. Cl. A61F 11/06; G10K 11/16; H03B 29/00

(52) U.S. Cl. 381/71.4; 381/71.8

(57) ABSTRACT

The present application describes a device for active noise reduction in the range of a passenger seat of an airplane or a land-based or water-based vehicle, respectively, the elements of which are either integrated into the passenger seat, or are installed in the immediate vicinity thereof. In the immediate vicinity of the head region of a passenger seat, at least one measurement microphone is disposed, the measurement signal of which is lead to a signal processing device. An output signal in counter-phase and amplified with respect to the measurement signal is generated there, serving for driving a reproduction device disposed in the vicinity of the head region of the passenger seat. At least one monitoring microphone is provided in the vicinity of the head region of the passenger seat, the output signal of which is lead to a further input of the signal processing device.

### FIELD OF THE INVENTION

The present invention relates to a device for active noise reduction, an arrangement of a device for active noise reduction in a passenger aircraft and to a method of actively reducing a noise in an aircraft.

### BACKGROUND OF THE INVENTION

During the flight, the passenger cabin of commercial airplanes is subject to various emissions of noise. Especially at long distance flights, wherein during the flight period there are also provided planed rest periods for the passengers, the continuous emission of noise may be perceived as disturbing.

In this context, noises are created by the propulsion motors, such as jet engines, by assisting aggregates as for example hydraulic actuators, by noises of wind of the cell and the airfoils, and by noises of flow of the air conditioner. The noise emission varies from airplane type to airplane type. Even within a model range of the same type, different types of emission may occur.

The sources of noise emission are divided into air sound and structure-borne sound. Since the isolating materials built into the cell especially serve to improve the isolation of heat, these possess a small damping factor against noise emission.

As is well known, the noise isolation in commercial airplanes is a compromise between effectivity of the sound isolation measures and the weight of these measures. A damping of structure-borne sound is hardly possible with the built-in isolating materials.

### SUMMARY OF THE INVENTION

According to an exemplary embodiment of the present invention, in immediate vicinity to the head region of a passenger seat, at least one measurement microphone is disposed, the measurement signal of which is lead to a signal processing device generating an output signal, with respect to the measurement signal of opposite phase and amplified, which serves for driving a reproduction device disposed in the vicinity of the head region of the passenger seat, wherein at least one monitoring microphone is provided in the vicinity of the head region of the passenger seat, the output signal of which is fed to a further entrance of the signal processing device.

It is believed that the level of noise in the region of the passenger seats, especially in the head region, may be lowered considerably, wherein the transmission of audio signals to the passenger may not be limited, and wherein the installation is light weighted.

By aid of the device according to an exemplary embodiment of the invention, without further measures in the range of the passenger, especially in the range of the head, a reduction of noise of up to 10 dB(A) may be reached. This corresponds to about cutting in half the noise subjectively perceived by the passenger.

The digitising of the analog measurement signals and the subsequent digital filtering of these signals is advantageous, so that from the picked up frequency spectrum may be extracted the frequencies which are typical for special sources of disturbing noise. The result of this frequency analysis may then used for controlling the circuit parts which finally create the signal in counter-phase to the disturbing noise.

The output signal of the device for active noise reduction may have an audio signal like for example announcements or music admixed to it, without resulting disturbance noise.

As the reproduction device for active noise reduction, various solutions are possible. To the one hand, the utilisation of a headphone-output disposed at the passenger seat presents itself, to the other hand, a loudspeaker disposed in the range of the passenger seat may be used. Accordingly, loudspeakers already installed in the cabin may be used for the radiation of anti-sound manifests itself. Thus, the increase of weight due to the installation of the device according to the invention for active noise reduction may be extremely small. Moreover, this device may easily be integrated into existing cabin systems, and the retrofitting into already existing airplanes or vehicles may be possible without problem.

A further embodiment of the reproduction device is its integration into the existing cabin casing or cladding or a carbody casing. This is especially true in the case that a signal generator is used, which due to its area-like construction may easily be adapted to given cabin casing forms, or be integrated therewith.

A further embodiment consists in providing at the passenger seat a device, for example in form of a regulation controller, by means of which the passenger may individually influence the device for active noise reduction.

The use of the device according to the invention for active noise reduction in the range of a passenger seat in an airplane or vehicle may be advantageous, in particular in planes with jet engines. However, the present invention may also be applied in any land-based and water-based vehicles.

### BRIEF DESCRIPTION OF THE DRAWINGS

Embodiment examples of the invention, with further embodiment details, are shown in the drawings in schematically simplified form, and will subsequently be described in more detail.

FIG. 1 shows a block diagram of a device for active noise reduction.

FIG. 2 shows a block diagram of an expanded circuit according to FIG. 1.

FIG. 3 shows a cut of an airplane-cabin having a device for active noise reduction.

FIG. 4 shows a passenger seat having functional elements of a device for noise reduction.

### DETAILED DESCRIPTION OF EXEMPLARY EMBODIMENTS

In principle, the following thought is basic to the device according to an exemplary embodiment of the invention. Each disturbing noise, at least to a certain extent, can be erased by a negative-feedback vibration of the same frequency. This anti-sound has to encounter the disturbing noise without a time delay, in counter-phase and with the same amplitude. Ideally, the disturbing sound is thereby completely erased.

In practice, a complete erasure of the disturbing sound will not occur, since the spectrum of the disturbing sound is combined from a variety of frequencies. A reduction of the local disturbing sound by 10 dB(A) may be realised. This means cutting in half the loudness of the disturbing noise nonetheless. For example, in order not to include in the noise reduction announcements or presentations of music in the range of the passenger, the portion of the disturbing frequencies has to be analysed. Only these extracted frequency parts may be used for the generation of the anti-sound.

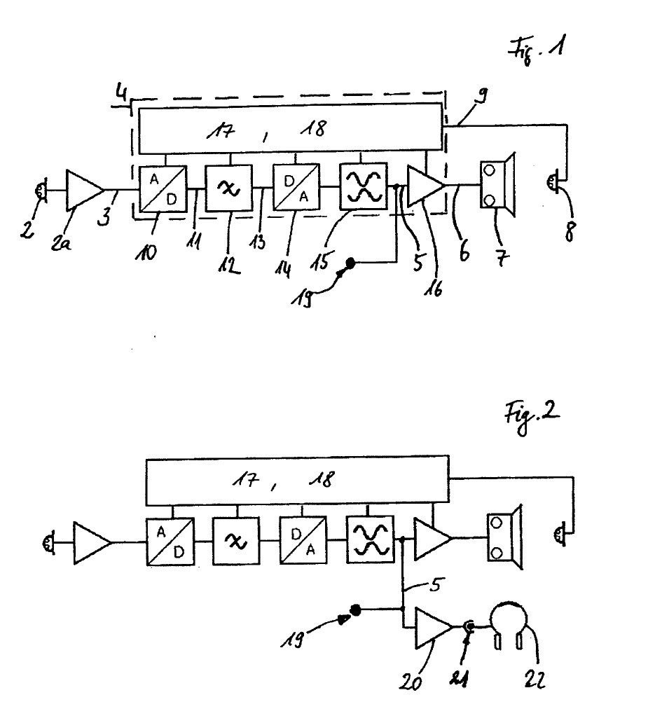

In FIG. 1, a block diagram of a device according to an exemplary embodiment of the invention for active noise reduction in the range of a passenger seat is shown. It includes a signal processing device 4, the input signal 3 of which stems from a measurement microphone 2. By means of a pre-amplifier 2a, the microphone signal may also have been amplified to the amplitude height necessary as input value for the signal processing device 4. In case of a condensor microphone, the pre-amplifier 2a also serves for feeding in direct voltage at the same time. The measurement microphone 2 detects the disturbing sound. To this end, it is disposed in the vicinity of the passenger.

The output signal 3 of the microphone 2 is fed to the analog-digital-converter 10 as input value. The A/D-converter 10 converts the analog signals into digital signals 11, to be able to hand these over to the subsequent digital filter 12. In the digital filter 12, the wanted signals are separated from the disturbing signals.

The control of the digital filter 12 is carried out by the signal processing device 4 which, to this end, contains a circuit for frequency analysis 17 and a signal processor 18. The filtered-out disturbance signals 13 are subsequently converted into analog signals in a digital-analog-converter 14, and are immediately fed into a 180 degree phase shifter 15 as input value. By means of the phase rotation, at the output of the phase shifter 15 there occurs the signal 5 in counter-phase to the input signal of the signal processing circuit 4, the so-called anti-sound signal. By means of an amplifier 16, the amplitude adjustment of the anti-sound signal is effected, so that the amplified output signal 6 may eventually be lead to a reproduction device 7, for example a loudspeaker, which usually is mounted in the vicinity of the passenger seat, and is directed towards the passenger. The control of amplitude of the anti-sound signal 6 is carried out by the digital signal processor 18.

Further, disposed in the region of the passenger, there is provided a monitor microphone 8, the output signal of which leads to a further input of the signal processing device 4. This control microphone monitors the continuous noise reduction. Thereby, positive feedbacks, which might result in unintended vibrations, may be avoided as well. The digital signal processor 18 therefore, in a controlling manner, accesses one or more of the further devices of the signal processing device 4. These are the analog-digital-converter 10, the digital filter 12, the digital-analog-converter 14, the phase-shifter 15 and finally the amplifier 16. Therefore, the signal processor 18 supervises the whole regulation and/or control loop.

In FIG. 1, there is further provided an audio input signal 19. This audio signal is representative for various wanted signals, as for example announcements or music presentations which shall not be influenced by the noise reduction. Therefore, these audio signals are mixed with the small band filtered counter-phase anti-sound signals 5. Thereby, the wanted signals are influenced only to an imperceptible extent.

A variant of this is shown in FIG. 2. The wanted signal 19 is again mixed with the anti-sound signal 5, and additionally lead to an amplifier 20. Via the plug-connector 21 disposed at the passenger seat, a headphone 22 is connected. Therefore, the passenger may enjoy video-play-ins or music without being disturbed. When the audio source is turned off, the passenger perceives a very low noise level.

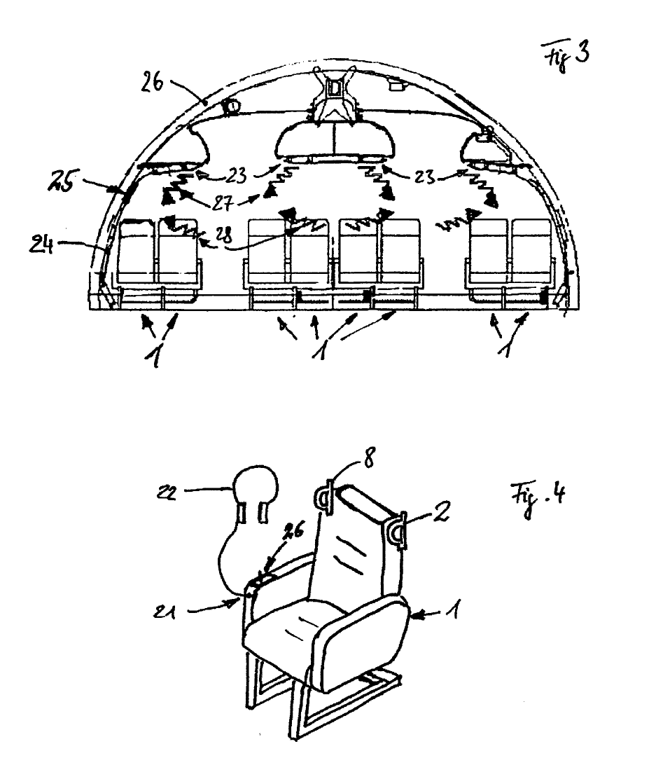

FIG. 3 shows a simplified section through an airplane cabin 26, in which a number of passenger seats 1 are installed. In this cabin, during flight operation, a number of disturbing signals occur, that may be measured in the area of the passenger, especially in the area of its head, as a concrete spectrum of frequencies having a typical amplitude. This sum of disturbing noises is symbolically shown in FIG. 3 as a serrated arrow 28, and in the area of each seat occurs as a spectrum typical for the implementation position of the seat. With the aid of the device for active noise reduction according to the invention, in the already described manner, there are generated anti-sound signals 6 which are delivered in the direction of a passenger seat 1 by means of suitable reproduction devices 7. In airplanes, in a simple manner, the loudspeaker 23 provided adjacently to the seat illumination and the fresh air flow-out opening above the respective passenger seats may be used in a simple way. Thereby, a lot of unnecessary weight for additionally necessary reproduction devices may be saved. The respective generated and radiated anti-sound signal is symbolically shown in FIG. 3 by means of further serrated arrows 27.

As reproduction device 7, there are also possible other embodiments of signal generators. As an example, in FIG. 3 there is integrated a signal generator 25 into the cabin casing 24. In this context, especially suited are flatly constructed signal generators on piezo basis or thin area-shaped signal generators constructed as synthetics-foil, and which are excellently suited for local emission of anti-sound.

Finally, FIG. 4 in a simplified manner, shows a single passenger seat together with the devices which are necessary or useful for the device for active noise reduction according to the invention. The representation has an informative character only, so that variants with arrangements having the same effect are possible within the range of the present invention.

At the passenger seat 1, according to the example shown, the measurement microphone 2 and also the monitoring microphone 8 are disposed in the range of the head of the passenger or the head rest. Both may be integrated into the passenger seat 1 in an unobstrusive manner. Thereby, the disturbing sound is detected as close to the head of the passenger as possible, and also the result of the noise reduction is examined. Further, at the passenger seat, for example at the arm rest, there is integrated the plug connection 21 for the head phone 22. This corresponds to the type of passengers seats which is customary nowadays, so that also the retrofitting of a device according to the invention for active noise reduction in this respect does not mean a big effort. Also, in the range of the arm rest, a device 26 for actively influencing the active noise reduction by the passenger is built-in. This installation as well does not mean any substantial effort, since at the installation place, control elements for other functions are present anyway.

In summary, it shall be noted that the device according to the invention for active noise reduction may be suited for installation into the passenger cabin of an airplane, in particular for airplanes with jet engines, the noise of which may very effectively be suppressed or reduced. Nonetheless, it is also possible to bring this device to use in a vehicle, wherein the necessity of a local noise reduction proves to be present. In this context, vehicle means not only a land-based vehicle, but water vehicles are possible as place of installation in the same manner.

It should be noted that the term “comprising” does not exclude other elements or steps and the “a” or “an” does not exclude a plurality. Also elements described in association with different embodiments may be combined.

It should also be noted that reference signs in the specification or in the claims shall not be construed as limiting the scope of the invention.

1. Device for active noise reduction in a proximity of a passenger seat in a passenger aircraft, in particular an aircraft with a jet engine, wherein the passenger seat has a head region, the device comprising:
at least one measurement microphone for arrangement in immediate vicinity of the head region of the passenger seat;
a signal processing device;
wherein a measurement signal generated by the at least one measurement microphone is input to the signal processing device;
wherein the signal processing device generates a first output signal which is amplified and in counter-phase to the measurement signal;
a reproduction device for arrangement in the vicinity of the head region of the passenger seat;
wherein the first output signal is input to the reproduction device for driving the reproduction device;
a monitoring microphone for arrangement in the vicinity of the head region of the passenger seat;
wherein the monitoring microphone is adapted for generating a second output signal which is input to the signal processing device.

2. The device for active noise reduction of claim 1, further comprising:
an analog-digital-converter provided at an input side of the signal processing device;
a digital filter; and
a digital-analog-converter
wherein the analog-digital-converter generates a third output signal which is input to the digital filter;
wherein a fourth digital output signal of the digital filter passes through the digital-analog-converter and is then used for generating a fifth counter-phase, amplified output signal.

3. The device for active noise reduction of claim 2,
wherein the signal processing device has a device for frequency analysis and a signal processor connected therewith;
wherein the signal processor and the device for frequency analysis, in a controlling manner, access at least one of the analog-digital-converter, the digital Filter, the digital-analog-converter, a phase shifter and a power amplifier.

4. The device for active noise reduction of claim 3,
wherein the device for frequency analysis detects at least one of frequencies and frequency ranges specific for sources of noise and controls the digital filter in such a manner that only the detected at least one of frequencies and frequency ranges are further processed for active noise reduction.

5. The device for active noise reduction of claim 3,
wherein a sixth output signal of the phase shifter is mixed with an audio signal.

6. The device for active noise reduction of claim 5,
wherein the sixth output signal is input via an amplifier to a connector for a headphone disposed at the passenger seat.

7. The device for active noise reduction of claim 1,
wherein the reproduction device is at least one loudspeaker disposed in the proximity of the passenger seat.

8. The device for active noise reduction of claim 1,
wherein the reproduction device is provided as a signal generator for arrangement in the proximity of the passenger seat in the range of a cabin casing of the passenger aircraft.

9. The device for active noise reduction of claim 8,
wherein the signal generator is arranged for being disposed in the cabin casing in an area-like manner.

10. The device for active noise reduction of claim 1, further comprising:
a device for active influencing of the active noise reduction for provision at the passenger seat.

11. Arrangement of the device of claim 1 in the proximity of a passenger seat in a passenger aircraft.

12. Method of actively reducing a noise in a proximity of a passenger seat in a passenger aircraft, the method comprising the steps of:
generating a measurement signal;
generating a first output signal which signal is in counter-phase to the measurement signal and which is amplified in comparison to the measurement signal;
driving a reproduction device by inputting the first output signal to the reproduction device;
generating a second output signal by means of a monitoring microphone an inputting the second output signal to the signal processing device for generating the first output signal.
